(19) United States
(12) Patent Application Publication
(10) Pub. No.: US 2026/0272554 A1
(43) Pub. Date: Sep. 17, 2026

(54) METHOD AND APPARATUS FOR PROVIDING IMPLANT FINE-ADJUSTMENT GUIDE

(71) Applicant: OSSTEM IMPLANT CO., LTD., Seoul (KR)

(72) Inventors: Sang Hyeong CHO, Gimpo-si (KR); Kyoo Ok CHOI, Seoul (KR)

(73) Assignee: OSSTEM IMPLANT CO., LTD., Seoul (KR)

(21) Appl. No.: 19/473,259

(22) PCT Filed: Apr. 25, 2024
(86) PCT No.: PCT/KR2024/005624

§ 371 Date: Oct. 7, 2025

(30) Foreign Application Priority Data
Jun. 28, 2023 (KR) 10-2023-0083257

(51) Int. Cl. A61B 34/10; A61C 8/00; G06F 3/04845

(52) U.S. Cl. CPC A61B 34/10; A61C 8/0018; G06F 3/04845; A61B 2034/102; A61B 2034/108

(57) ABSTRACT

Disclosed are a method for providing an implant adjustment guide and an apparatus to which the method is applied. The method for providing an implant adjustment guide, according to an embodiment of the present disclosure, may comprise the steps of: obtaining an oral cavity shape of a person in which an implant is to be placed; determining, by using the oral cavity shape, an initial position of a graphic object of the implant; determining, by using the oral cavity shape, an adjustable region for the graphic object of the implant, wherein the adjustable region is a region adjacent to the initial position of the implant, is a maximum region where, according to the oral cavity shape, no object is present inside the oral cavity, and is a region displayed at a fixed position regardless of positional movement of the graphic object of the implant; and providing an implant fine-adjustment screen including the graphic object of the implant and an adjustable region graphic object in which the determined adjustable region is visualized.

### TECHNICAL FIELD

The present disclosure relates to a method for providing an implant fine-adjustment guide and a device to which the method is applied.

### BACKGROUND ART

An implant simulation program is provided that proposes the size and orientation of an implant to be placed in the oral cavity of a patient prior to performing an implantation procedure.

When performing the procedure of placing an implant in the oral cavity of a patient, the operator must maintain a minimum distance between the implant to be placed and adjacent teeth located around the intended placement position, pre-placed implants located around the position, and implants to be placed later, and the implant must be placed only at a location where a drilling hole for generating a surgical guide can be formed. Accordingly, the above-described implant simulation program needs to provide the operator with guidance regarding appropriate constraints to be recognized in fine adjustment of the implant position.

Nonetheless, the interface of a conventional implant simulation program, which displays the shape of the implant to be placed and the inside of the patient's oral cavity, ensures freedom for the user of the implant simulation program, thereby supporting the user to freely determine the position or orientation of the implant shape in 2D or 3D images.

As described above, if there is no restriction range for adjustment of the size, position, or orientation of the implant shape, collisions may occur between anatomical structures in the oral cavity and adjacent objects during actual implantation. Therefore, when finely adjusting the position of the implant, there is a demand to provide guidance that can intuitively inform the operator of the limits of the adjustment.

### DISCLOSURE

### Technical Problem

An object to be achieved through some embodiments of the present disclosure is to provide a method for providing an implant fine-adjustment guide that assists a user in easily recognizing constraints on adjusting the size, position, or orientation of an implant to be placed, and an apparatus for performing the method.

Another object to be achieved through some embodiments of the present disclosure is to provide a method for providing an implant fine-adjustment guide that assists a user in easily recognizing a region where collision with an adjacent implant may occur during a process of placing an implant, and an apparatus for performing the method.

Another object to be achieved through some embodiments of the present disclosure is to provide a method for providing an implant fine-adjustment guide that assists a user in easily recognizing a collision when a collision occurs with another object while an implant shape to be placed is being adjusted in size, moved, or rotated, and an apparatus for performing the method.

The objects of the present invention are not limited to those mentioned above, and other objects not mentioned will be clearly understood by those skilled in the art from the description below.

### Technical Solution

A method for providing an implant adjustment guide according to an embodiment of the present disclosure may include the steps of: obtaining an oral cavity shape of a subject undergoing implant placement; determining, by using the oral cavity shape, an initial position of a graphic object of the implant; determining, by using the oral cavity shape, an adjustable region for the graphic object of the implant, wherein the adjustable region, which is a region adjacent to the initial position of the implant, is a maximum region where, according to the oral cavity shape, no object is present inside the oral cavity, and is a region displayed at a fixed position regardless of positional movement of the graphic object of the implant; and providing an implant fine-adjustment screen including the graphic object of the implant and an adjustable region graphic object in which the determined adjustable region is visualized.

In some embodiments, the step of determining, by using an oral cavity shape, an adjustable region for the graphic object of the implant may include the step of obtaining a region spaced apart from an intraoral object by a reference value in the oral cavity shape.

In some embodiments, the step of obtaining a region spaced apart from an intraoral object by a reference value in the oral cavity shape may include the steps of obtaining a shape of the intraoral object, and obtaining a region spaced apart from an outer contour line of the intraoral object by a predefined reference value.

In some embodiments, the step of obtaining a region spaced apart from an outer contour line of the intraoral object by a predefined reference value may include the step of, when an uppermost distance between the outer contour line of the intraoral object and the implant to be placed is minimum, determining, as a maximum width of the adjustable region, a distance obtained by excluding a distance that must not be invaded by the implant to be placed from the uppermost distance.

In some embodiments, the step of providing an implant fine-adjustment screen including the graphic object of the implant and an adjustable region graphic object in which the determined adjustable region is visualized may include the steps of: receiving an implant adjustment signal generated by a user input to a user interface included in the implant fine-adjustment screen; moving a graphic object position of the implant in response to the implant adjustment signal; and outputting a predefined user confirmation message when the moved graphic object position of the implant has deviated from the adjustable region.

In some embodiments, the step of moving a graphic object position of the implant in response to the implant adjustment signal may include the step of, when the moved graphic object position of the implant has deviated from the adjustable region, providing an implant fine-adjustment screen in which the graphic objects of the implant are respectively arranged at a position before movement and a position after movement according to the implant adjustment signal.

In some embodiments, the step of providing an implant fine-adjustment screen in which the graphic objects of the implant are respectively arranged at a position before movement and a position after movement according to the implant adjustment signal may include the step of providing the implant fine-adjustment screen such that the graphic object of the implant at the position after movement according to the implant adjustment signal is overlaid on the graphic object of the implant at the position before movement.

In some embodiments, the step of, when the moved graphic object position of the implant has deviated from the adjustable region, providing an implant fine-adjustment screen in which the graphic objects of the implant are respectively arranged at a position before movement and a position after movement according to the implant adjustment signal may include the step of generating a selection message regarding whether to apply the implant adjustment signal.

In some embodiments, the step of, when the moved graphic object position of the implant has deviated from the adjustable region, providing an implant fine-adjustment screen in which the graphic objects of the implant are respectively arranged at a position before movement and a position after movement according to the implant adjustment signal may include the step of changing a region expression element of the graphic object of the implant.

In some embodiments, the step of moving the graphic object position of the implant in response to the implant adjustment signal may include the steps of: obtaining a required guide drilling hole region; obtaining a crown region of an adjacent tooth of the implant in the oral cavity shape; obtaining a region in the oral cavity shape which is spaced apart from the crown region by a reference value and in which the required guide drilling hole region is entirely included; and selecting, as an implant adjustable region, the region in the oral cavity shape which is spaced apart from the crown region by the reference value and in which the required guide drilling hole region is entirely included.

According to another embodiment of the present disclosure, a system for providing an implant adjustment guide includes a memory into which an implant adjustment guide providing program is loaded and a processor configured to execute the implant adjustment guide providing program, wherein the implant adjustment guide providing program comprises: an instruction for obtaining an oral cavity shape of a subject undergoing implant placement; an instruction for determining, by using the oral cavity shape, an initial position of a graphic object of an implant; an instruction for determining, by using the oral cavity shape, an adjustable region for the graphic object of the implant, wherein the adjustable region, which is a region adjacent to the initial position of the implant, is a maximum region in which, according to the oral cavity shape, no object is present inside the oral cavity, and is a region displayed at a fixed position regardless of positional movement of the graphic object of the implant; an instruction for providing an implant fine-adjustment screen including the graphic object of the implant and an adjustable region graphic object in which the determined adjustable region is visualized; an instruction for receiving an implant adjustment signal generated by a user input to a user interface included in the implant fine-adjustment screen; an instruction for moving a graphic object position of the implant in response to the implant adjustment signal; and an instruction for outputting a predefined user confirmation message when the moved graphic object position of the implant has deviated from the adjustable region.

### Effects of the Invention

According to some embodiments of the present disclosure, it is possible to prevent collisions with separate intraoral objects that may occur during adjustment of the size of an implant to be placed, movement thereof, or rotation thereof by simulating a process of performing implant placement.

According to some embodiments of the present disclosure, factors to be considered before implant placement may be displayed on a user interface during an implant placement simulation process, so that an implant placement operator may intuitively set the size, position, and orientation of an implant shape that does not collide with separate intraoral objects.

The effects of the present invention are not limited to the aforementioned effects, and other effects not mentioned will be clearly understood by those skilled in the art from the description below.

### DESCRIPTION OF DRAWINGS

FIG. 1 is an exemplary block diagram to which an implant adjustment guide providing system according to an embodiment of the present disclosure may be applied.

FIG. 2 is a flowchart illustrating a method for providing an implant adjustment guide according to some embodiments of the present disclosure.

FIGS. 3 to 5 are flowcharts for describing in more detail the method for providing an implant adjustment guide described with reference to FIG. 2.

FIG. 6 is a view illustrating a manipulator configured to receive a control signal from an implant placement operator in some embodiments of the present disclosure.

FIGS. 7 to 10 are views for describing a step of obtaining an adjustable region by using an object around a position where an implant to be placed is to be placed, which may be performed in some embodiments of the present disclosure.

FIGS. 11 to 13 are views for describing a step of determining an adjustable region by using a crown region of an adjacent tooth of an implant to be placed, which may be performed in some embodiments of the present disclosure.

FIG. 14 is a view illustrating an implant fine-adjustment screen generated when a graphic object of an implant deviates from an adjustable region upon application of an adjustment signal, which may be implemented in some embodiments of the present disclosure.

FIG. 15 is a hardware configuration diagram of a computing device to which an implant simulation program may be applied according to another embodiment of the present disclosure.

### MODE OF THE INVENTION

Hereinafter, example embodiments of the present disclosure will be described in detail with reference to the accompanying drawings. The advantages and features of the present disclosure and the manner of achieving the advantages and features will become apparent with reference to embodiments described in detail below together with the accompanying drawings. However, the present disclosure may be implemented in many different forms and should not be construed as being limited to the embodiments set forth herein, and the embodiments are provided such that this disclosure will be thorough and complete and will fully convey the scope of the present disclosure to those skilled in the art, and the present disclosure is defined only by the scope of the appended claims.

In describing the present disclosure, if it is determined that a detailed description of a related known configuration or function may obscure the gist of the present invention, the detailed description will be omitted.

Unless otherwise defined, all terms used in the present specification (including technical and scientific terms) may be used in a sense that can be commonly understood by those skilled in the art, but the terms may vary according to the intention of those of ordinary skill in the art, precedents, or new technology in the art. The terminology used herein is for the purpose of describing particular embodiments only and is not intended to be limiting of the disclosure.

In this specification, the singular also includes the plural unless specifically stated otherwise in the phrase. Additionally, plural expressions include singular concepts unless the context clearly specifies that they are plural.

In In addition, in describing the component of this disclosure, terms, such as first, second, A, B, (a), (b), can be used. These terms are only for distinguishing the components from other components, and the nature or order of the components is not limited by the terms.

Hereinafter, various embodiments of the present disclosure will be described in detail with reference to the accompanying drawings.

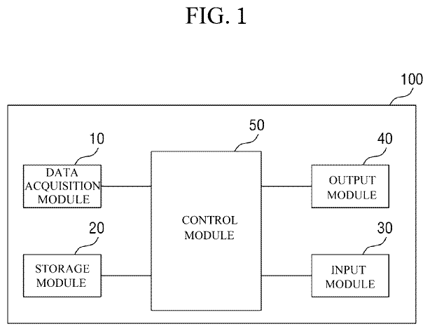

Hereinafter, the configuration and operation of an implant adjustment guide providing system according to an embodiment of the present disclosure will be described with reference to FIG. 1.

The implant adjustment guide providing system may be configured as one or more computing devices that provide a dental implant placement operator with the functions of obtaining an oral cavity shape of a patient for dental treatment, adjusting properties related to implant placement such as the size, position, or orientation of an implant to be placed in the patient's oral cavity, and establishing an implant placement plan through simulation.

That is, the implant adjustment guide providing system 100 may be configured as a single computing device, or may be configured as a plurality of computing devices. For example, a data acquisition module 10, a storage module 20, and a control module 50 shown in FIG. 1 may be included in a server device, and an input module 30 and an output module 40 may be included in a user terminal. In this case, the server device may be configured by using one or more physical servers included in a server farm based on cloud technology such as a virtual machine. In addition, all of the data acquisition module 10, the storage module 20, the input module 30, the output module 40, and the control module 50 shown in FIG. 1 may be included in a user terminal.

The implant adjustment guide providing system 100, by displaying on the output module 40 a range in which fine adjustment of an implant to be placed is possible using information about intraoral objects that may collide with the implant, assists the implant placement operator in clearly recognizing a safe region in which the implant placement may be performed.

The implant may refer to an artificial structure that is placed inside the gum to replace a tooth.

The storage module 20 may store oral cavity image data of an implant subject undergoing implant placement. The oral cavity image data may be computed tomography (CT) images, X-ray images, or scan data representing a three-dimensional structure inside the oral cavity of the implant subject. The oral cavity image data may be data generated by an oral cavity image capturing device (not shown), received from the oral cavity image capturing device by the implant adjustment guide providing system 100, and stored in the storage module 20.

The data acquisition module 10 may obtain the oral cavity image data. In some embodiments, the data acquisition module 10 may perform preprocessing image processing on the oral cavity image data. For example, the preprocessing image processing may include segmentation of a tooth region, sharpening, or contour extraction. The data acquisition module 10 may generate data of an oral cavity shape from the oral cavity image data through the preprocessing image processing.

The oral cavity shape may include position information of each object inside the oral cavity of the implant subject, and the objects inside the oral cavity may include teeth or pre-placed implants. Hereinafter, the objects inside the oral cavity will be referred to as intraoral objects.

The control module 50 may determine an initial position for an implant to be placed by using the oral cavity shape. For example, the control module 50 may determine the initial position of the implant by inputting a tooth number of a missing tooth corresponding to the implant into an initial position determination algorithm or an initial position determination AI model.

The control module 50 may determine an adjustable region for the implant to be placed by using the oral cavity shape. For example, the control module 50 may determine the adjustable region by using the oral cavity shape and the initial position of the implant.

The adjustable region may mean an adjacent region of the initial position of the implant in which no intraoral object exists. As described above, the intraoral object may include a tooth or a pre-placed implant. Accordingly, the adjustable region may be an adjacent region of the initial position of the implant in which no tooth or pre-placed implant exists. That is, the adjustable region may be understood as a region in which the position of the implant can be adjusted without a collision with an intraoral object of the implant subject, and as a region in which fine adjustment of the implant at the initial position is possible.

Additionally, the control module 50 may provide an implant fine-adjustment screen including an adjustable region graphic object in which the adjustable region is visualized and a graphic object of the implant. That is, the implant placement operator may intuitively recognize, through the implant fine-adjustment screen, a range in which fine adjustment of the graphic object of the implant placed at the initial position is possible. In other words, the adjustable region graphic object included in the implant fine-adjustment screen may be understood as performing a guide role for implant adjustment.

As described above with reference to FIG. 1, the configuration and operation of the implant adjustment guide provision system according to this embodiment have been explained. An operating method of the implant adjustment guide providing system 100 according to this embodiment will be more clearly understood by reference to other embodiments to be described below. In addition, a technical idea that can be understood through the above-described embodiments of the implant adjustment guide providing system according to this embodiment may be reflected in other embodiments to be described below, even if not separately specified.

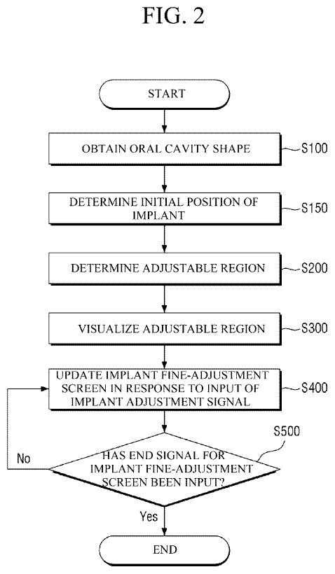

Hereinafter, a method for providing an implant adjustment guide according to another embodiment of the present disclosure will be described with reference to FIGS. 2 to 5. The method for providing an implant adjustment guide according to this embodiment may be performed by a computing device. In addition, in the method for providing an implant adjustment guide according to this embodiment, some operations may be performed by a first computing device, and the remaining operations may be performed by a second computing device. For example, some operations of the method for providing an implant adjustment guide according to this embodiment may be performed by a computing device installed at a treatment site such as a dental clinic, and the remaining operations may be performed by a cloud compute instance. Hereinafter, when a subject performing each operation is omitted, it may be understood that the subject performing the operation is the computing system.

Hereinafter, the description will be made with reference to FIG. 2.

In step S100, oral cavity image data of an implant subject is obtained, and data of an oral cavity shape may be obtained from the oral cavity image data. As described above, the data of the oral cavity shape may be obtained by using results of preprocessing image processing such as segmentation of a tooth region on the oral cavity image data.

Next, in step S150, an initial position for a graphic object of an implant to be placed may be determined. The initial position may be determined by using the data of the oral cavity shape obtained in step S100, or by using both the data of the oral cavity shape and a tooth number of the implant to be placed input from a user.

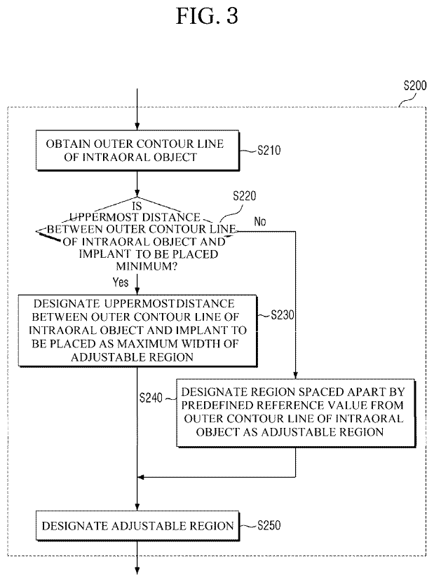

Next, in step S200, an adjustable region may be determined. At this time, an outer contour line of an intraoral object included in the oral cavity shape of the implant subject is obtained, and a region spaced apart from the outer contour line by a distance greater than or equal to a clearance distance, which is a predefined reference value, may be determined as the adjustable region. In addition, the adjustable region may mean a region in which adjustment such as movement, rotation, or specification change of the graphic object of the implant to be placed at the initial position determined in step S150 is allowed. Step S200 will be described in more detail below with reference to FIG. 3.

The clearance distance is a distance that must not be invaded by the graphic object of the implant to be placed and is a range partially overlaid on the outer contour line of the intraoral object. For example, since the implant must be placed at a position spaced apart by at least about 1.5 mm from an adjacent tooth at the intended placement position, the clearance distance to the adjacent tooth is about 1.5 mm.

Next, in step S300, an implant fine-adjustment screen including an adjustable region graphic object and the graphic object of the implant may be displayed on a terminal device of the implant placement operator. The adjustable region may be visualized such that it is visually distinguished and recognized from surrounding regions by being expressed differently from the surrounding regions in color, shading, or shape. That is, the adjustable region graphic object for visualization may be included in the implant fine-adjustment screen. As a result of the visualization, the implant placement operator may intuitively recognize the adjustable region.

Next, in step S400, an implant adjustment signal is input, and the implant fine-adjustment screen may be updated in response to the input implant adjustment signal. For example, the implant adjustment signal may be a signal generated by manipulation of a manipulator included in the implant fine-adjustment screen. The manipulator included in the implant fine-adjustment screen may receive adjustment signals from the implant placement operator regarding movement, size change, rotation, or crown model change of the implant graphic.

When the graphic object of the implant updated according to the input adjustment signal has deviated from the adjustable region, an implant fine-adjustment screen including a deviation signal of the adjustable region and a selection message regarding whether to apply the adjustment signal of the implant may be generated.

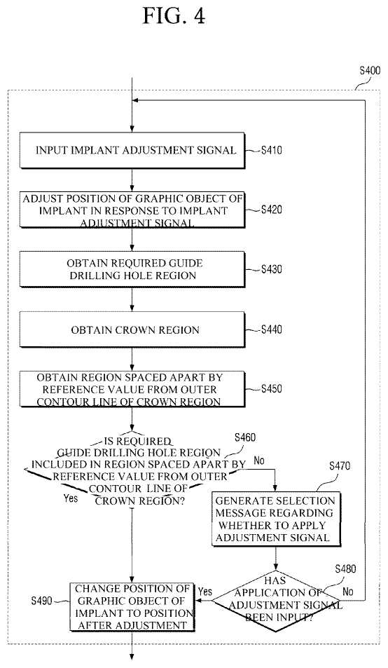

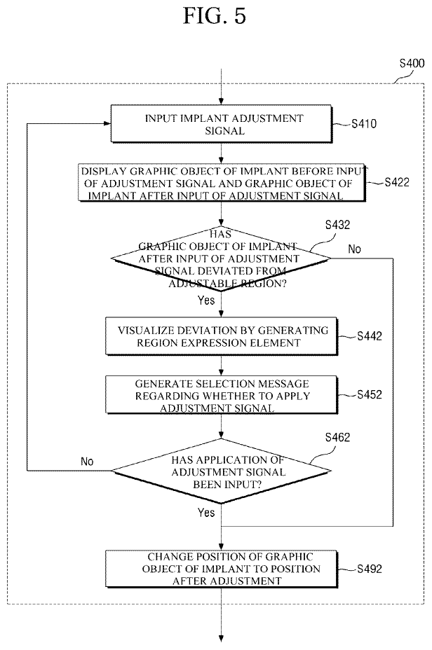

Even when an outer contour line of an adjacent tooth of the graphic object of the implant updated according to the input adjustment signal fails to include a guide drilling hole required region of the implant to be placed, an implant fine-adjustment screen including a selection message regarding whether to apply the adjustment signal of the implant may be generated. Step S400 will be described in more detail below with reference to FIGS. 4 and 5.

Next, in step S500, an end signal for the implant fine-adjustment screen may be input. When the end signal for the implant fine-adjustment screen is input, the manipulator is deactivated, and the implant graphic included in the updated implant fine-adjustment screen may be stored. The end signal for the implant fine-adjustment screen may be a signal generated according to a selection input to a screen end graphic object included in the implant fine-adjustment screen.

If the end signal for the implant fine-adjustment screen is not input, the activation state of the manipulator included in the implant fine-adjustment screen is maintained, and the process of receiving an implant adjustment signal corresponding to step S400 and updating the implant fine-adjustment screen according to the input adjustment signal may be repeated.

Hereinafter, step S200 of determining the adjustable region will be described in more detail with reference to FIG. 3.

In step S210, an outer contour line of the obtained intraoral object may be obtained. As described above, the intraoral object may include an object of a tooth inside the oral cavity of the implant subject or an object of a pre-placed implant. In addition, the outer contour line may refer to an outline of the intraoral object.

In step S220, it may be determined whether an uppermost distance between the outer contour line of the intraoral object and the graphic object of the implant to be placed is a minimum among distances between the graphic object of the implant to be placed and the adjacent tooth of the implant to be placed.

The uppermost distance may be a distance between the graphic object of the implant to be placed and the adjacent tooth of the implant to be placed at a gum surface. The distance between the graphic object of the implant to be placed and the adjacent tooth of the implant to be placed may be a distance between the graphic object of the implant to be placed spaced apart from the gum surface by a specific depth and the adjacent tooth of the implant to be placed spaced apart from the gum surface by a depth equal to the specific depth. Accordingly, the distance between the graphic object of the implant to be placed and the adjacent tooth of the implant to be placed may include one or more distances.

In other words, in step S220, it may be determined whether the uppermost distance among distances between the outer contour line of the adjacent tooth of the implant to be placed, which is one of the intraoral objects, and the graphic object of the implant to be placed is the minimum distance.

When the uppermost distance is determined to be the minimum distance, in step S230, a distance obtained by excluding a clearance distance to outer contour lines of adjacent teeth on both sides from the uppermost distance between the outer contour line of the adjacent tooth of the implant to be placed and the graphic object of the implant to be placed may be designated as a maximum width of the adjustable region. When the uppermost distance is determined to be the minimum distance, it may be considered that a degree of freedom of implant adjustment is low in performing implant placement. Therefore, the implant placement operator may be provided with a minimum width of the placement region so as not to input unnecessary adjustments or consider impossible adjustments.

When the uppermost distance is determined not to be the minimum distance, in step S240, a region spaced apart by a predefined reference value from the outer contour line of the adjacent tooth of the implant to be placed may be designated as the adjustable region. When the uppermost distance is determined not to be the minimum distance, it may be considered that a degree of freedom of implant adjustment is high in performing implant placement. Therefore, a maximum width of the placement region may be provided to the implant placement operator so as to ensure a degree of freedom of adjustment.

When the adjustable region is designated in step S250, an implant fine-adjustment screen including an adjustable region graphic object may be generated in step S300.

Next, step S400 of updating the implant fine-adjustment screen in response to input of the implant adjustment signal, as described with reference to FIG. 2, will be described in more detail with reference to FIG. 4.

In step S410, an implant adjustment signal may be input to the implant fine-adjustment screen including the adjustable region graphic object. The implant fine-adjustment screen may include a manipulator capable of receiving an implant adjustment signal from the implant placement operator.

In step S420, the implant fine-adjustment screen may be updated to include a version of an implant graphic object whose position, shape, or size has been adjusted according to the input implant adjustment signal.

Next, in step S430, a required guide drilling hole region necessary for actually placing the implant to be placed in the same manner as the implant graphic object of the version adjusted according to the input implant adjustment signal may be determined.

The required guide drilling hole region is a predetermined region required for a tool for implant placement and drilling to perform operation during implant placement. The required guide drilling hole region may be generated in a gum surface region between adjacent teeth of a tooth for which the implant is to be placed.

The required guide drilling hole region may be determined by using the implant graphic object of the version adjusted according to the implant adjustment signal. That is, the required guide drilling hole region of the implant to be placed may be a region corresponding to a placement direction of the implant graphic object of the version adjusted by reflecting the adjustment signal.

Next, in step S440, a crown region of the adjacent tooth of the implant to be placed is obtained, and in step S450, a region spaced apart by a reference value from an outer contour of the obtained crown region may be obtained.

The crown region of the adjacent tooth of the implant to be placed may be obtained from the oral cavity shape, and the outer contour of the crown region may be obtained from the outer contour line of the intraoral object.

In step S460, it may be determined whether the region spaced apart by the reference value from the outer line of the crown region may include the required guide drilling hole region.

When it is determined that the region spaced apart by the reference value from the outer line of the crown region is wide enough to include the required guide drilling hole region, in step S490, an implant fine-adjustment screen in which the position of the graphic object of the implant is changed to an adjusted position may be generated.

When it is determined that the region spaced apart by the reference value from the outer line of the crown region is not wide enough to include the required guide drilling hole region, the implant reflecting the adjustment signal cannot be placed, and therefore an implant fine-adjustment screen including a selection message regarding whether to apply the adjustment signal input may be generated in step S470.

In step S480, a selection regarding the selection message on whether to apply the adjustment signal generated on the implant fine-adjustment screen may be input. In one embodiment, a selection not to apply the adjustment signal may be input. When the selection not to apply the adjustment signal is input, the activation state of the manipulator included in the implant fine-adjustment screen is maintained, and the step of receiving an implant adjustment signal and updating the implant fine-adjustment screen according to the input adjustment signal, which corresponds to step S400, may be repeated.

In some embodiments, it may be determined whether or not the implant reflecting the adjustment signal is in a state in which placement is impossible, and when it is determined that placement of the implant reflecting the adjustment signal is impossible, a selection message for requesting a user's selection as to whether to apply the adjustment signal may be generated. When the selection to apply the adjustment signal is made, in step S490, an implant fine-adjustment screen in which the position of the graphic object of the implant is changed to a position to which the adjustment signal has been applied may be generated.

Next, as step S400 of updating the implant fine-adjustment screen in response to input of the implant adjustment signal described with reference to FIG. 2, another embodiment different from the embodiment described with reference to FIG. 4 will be described in more detail with reference to FIG. 5.

In step S410, the implant adjustment signal may be input to an implant fine-adjustment screen including the adjustable region graphic object.

Next, in step S422, an implant fine-adjustment screen including both a graphic object of the implant before receiving the adjustment signal and a graphic object of the implant after receiving the adjustment signal may be generated.

One or more graphic objects of the implant may be displayed such that an object reflecting the latest adjustment signal is displayed in a higher layer, and the object displayed in a higher layer may be expressed more distinctly in shape. The higher the layer in which the object is displayed, the more clearly the object is expressed, which may provide the effect of allowing the implant placement operator to first recognize and confirm the object reflecting the latest adjustment signal.

Next, in step S432, it may be determined whether the graphic object of the implant reflecting the adjustment signal has deviated from the adjustable region graphic object. When it is determined that the graphic object of the implant reflecting the adjustment signal is included in the adjustable region graphic object, in step S492, an implant fine-adjustment screen in which the position of the graphic object of the implant is changed to a position to which the adjustment signal has been applied may be generated.

When it is determined that the graphic object of the implant reflecting the adjustment signal has deviated from the adjustable region graphic object, in step S442, an implant fine-adjustment screen in which the adjustable region graphic object is visualized by using region expression elements may be generated.

When it is determined that the graphic object of the implant reflecting the adjustment signal has deviated from the adjustable region graphic object, the adjustable region graphic object included in the implant fine-adjustment screen may be expressed differently from surrounding regions using one or more expression elements including shape, color, and shading.

Next, in step S452, the implant fine-adjustment screen may be updated to an implant fine-adjustment screen including a selection message regarding whether to apply the adjustment signal.

In step S462, whether to apply the immediately input implant adjustment signal may be input through the selection message displayed on the implant fine-adjustment screen. When the selection not to apply the adjustment signal is input, the activation state of the manipulator included in the implant fine-adjustment screen is maintained, and the process of receiving an implant adjustment signal and updating the implant fine-adjustment screen according to the input adjustment signal, which corresponds to step S410, may be repeated.

Through the selection message displayed on the implant fine-adjustment screen, a selection to apply the adjustment signal may be input with recognition that the implant reflecting the adjustment signal cannot be placed. As the application of the adjustment signal is selected, in step S492, an implant fine-adjustment screen including a graphic object of the implant reflecting the adjustment signal may be generated.

In addition, the implant fine-adjustment screen including one or more graphic objects of the implant may receive, from the implant placement operator, a selection to re-change the adjustment conditions back to a previous shape without applying the adjustment signal. The implant fine-adjustment screen including a plurality of graphic objects of the implant may provide the implant placement operator with an efficient method of adjusting the graphic object of the implant by selecting one among a plurality of samples.

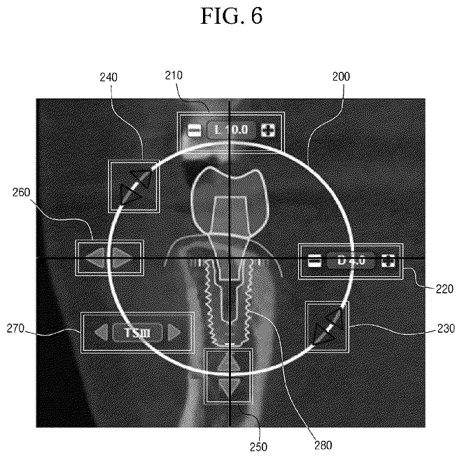

Next, a manipulator configured to receive input for an adjustment of a graphic object of an implant from the implant placement operator is described with reference to FIG. 6. A manipulator 200 shown in FIG. 6 may be activated when a selection for adjustment of the implant to be placed is input, and buttons may be arranged on an outline of the manipulator 200. As shown in FIG. 6, the manipulator 200 may be generated in a circular shape. The manipulator 200 may be overlaid on the adjustable region graphic object and included in the implant fine-adjustment screen. The manipulator 200 may receive input for an adjustment for each of an implant 280 and a crown.

The implant fine-adjustment screen may include, as buttons arranged on the outline of the adjustment manipulator, buttons 210, 220, and 270 for changing specifications of the implant by using a position of the implant to be placed and objects around a position where the implant is to be placed, rotation buttons 230 and 240 for adjusting an angle and a position of the implant to be placed, and movement buttons 250 and 260.

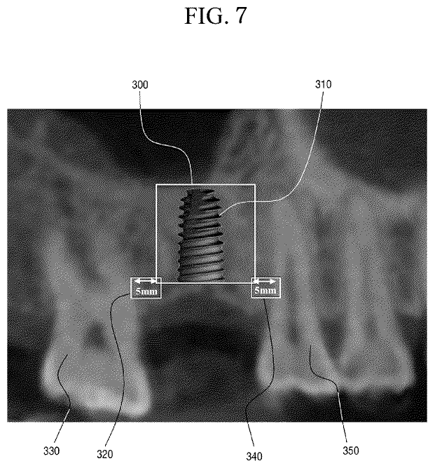

Next, with reference to FIGS. 7 to 10, operations related to determining an adjustable region by using an object around a position where the implant to be placed is to be placed will be described. The adjustable region may be generated only in a region spaced apart by a predetermined distance from an object around a position where the implant to be placed is to be placed. An adjustable region of an upper jaw may be generated only in a region spaced apart by a minimum distance from a maxillary sinus region, and an adjustable region of a lower jaw may be generated only in a region spaced apart by a minimum distance from a neural tube.

FIG. 7 illustrates an example of an adjustable region 300 in which an implant 310 to be laced is completely included. As shown in FIG. 7, among distances between a left adjacent tooth 330 and the implant 310 to be placed, a uppermost distance may correspond to a minimum distance, and among distances between a right adjacent tooth 350 and the implant 310 to be placed, a uppermost distance may correspond to a minimum distance. As in the example, when the uppermost distance among distances between the adjacent teeth and the implant to be placed is the minimum distance, the adjustable region graphic object may be generated in a rectangular shape having as a width a distance obtained by excluding clearance distances to outer contour lines of adjacent teeth on both sides from the uppermost distance. As shown in FIG. 7, when adjacent objects of the implant to be placed are both teeth, clearance distances 320 and 340 from the left adjacent tooth 330 and the right adjacent tooth 350, respectively, may be the same.

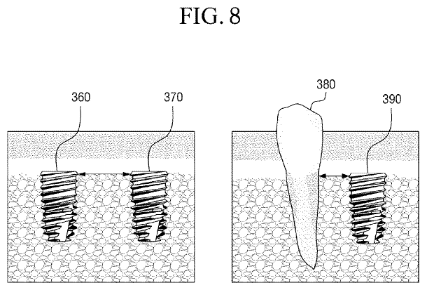

FIG. 8 illustrates an example in which an implant to be placed is placed adjacent to a pre-placed implant or an adjacent tooth at a placement position of the implant to be placed. As shown in FIG. 8, an implant 370 to be placed may be placed in a state spaced apart from a pre-placed implant 360 by a predefined implant clearance. In addition, an implant 390 to be placed may be placed in a state spaced apart from an adjacent tooth 380 by a predefined tooth clearance distance. As in the example, the clearance distance to be maintained between an intraoral object and an implant to be placed may vary depending on the type of intraoral object. A clearance distance between implants within the oral cavity may be longer than a clearance distance from an intraoral tooth.

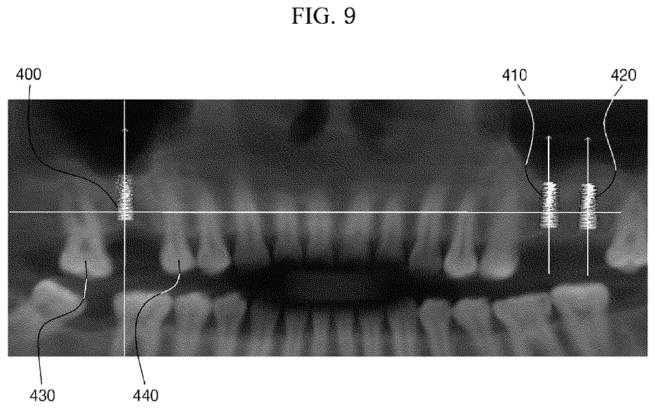

FIG. 9 illustrates an example in which a graphic object of a pre-generated implant or a graphic object of an adjacent tooth adjacent to a placement position of an implant to be placed and a graphic of the implant to be placed area generated adjacent to each other.

As shown in FIG. 9, a graphic object 420 of a pre-generated implant and a graphic object 410 of an implant to be placed may be displayed on the implant fine-adjustment screen in a state spaced apart by a predefined clearance distance between implants. An adjustable region of the graphic object 410 of the implant to be placed may be determined as a region spaced apart from the graphic object 420 of the pre-generated implant by the clearance distance between implants.

In addition, a graphic object 400 of the implant to be placed and adjacent teeth 430 and 440 may be displayed on the implant fine-adjustment screen in a state spaced apart by a distance greater than or equal to a predefined clearance distance from a tooth. An adjustable region of the graphic object 400 of the implant to be placed may be determined as a region spaced apart from the adjacent teeth 430 and 440 by the clearance distance from a tooth.

In addition, a spacing distance between the graphic object 400 of the implant to be placed and a left adjacent tooth 430 may not be the same as a spacing distance between the graphic object 400 of the implant to be placed and a right adjacent tooth 440.

As in the example, a minimum spacing distance between an intraoral object and an implant to be placed may vary depending on the type of intraoral object. However, the minimum spacing distance is predefined as a clearance distance, and as long as the clearance distance corresponding to the minimum criterion is satisfied, the remaining position adjustment is made according to what is input by the implant placement operator, and thus the distances between the left and right adjacent teeth and the implant to be placed may not necessarily be the same.

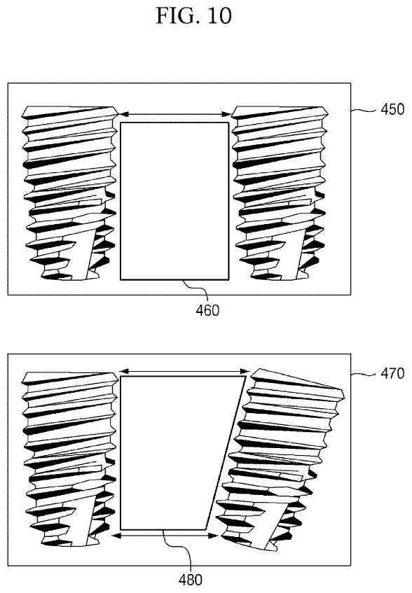

Next, the operation of obtaining the adjustable region by using an adjacent object of an implant to be placed will be described with reference to FIG. 10.

As shown at 450 of FIG. 10, the minimum distance among distances between outer lines of an implant to be placed and an adjacent object of the implant to be placed may be the uppermost distance. The distance between the outer lines of the implant to be placed and the adjacent object of the implant to be placed is the distance between the outer line of the adjacent object of the implant to be placed that is spaced apart by a specific depth from the gum surface and the outer line of the implant to be placed that is placed by the same depth from the gum surface.

When the minimum distance between the outer lines of the implant to be placed and the adjacent object of the implant to be placed is the uppermost distance, an adjustable region 460 may be generated in a rectangular shape having as a width a distance obtained by excluding clearance distances to outer lines of adjacent objects on both sides from the uppermost distance. The clearance distance is a distance between the adjustable region 460 and the adjacent object of the implant to be placed.

When the minimum distance among distances between the outer contours of the implant to be placed and the adjacent object of the implant to be placed is determined as the uppermost distance, it may be considered that a degree of freedom of implant adjustment is low in performing implant placement. Therefore, the implant placement operator may be provided with a minimum width of the placement region so as not to input unnecessary adjustments or consider impossible adjustments.

As shown at 470 of FIG. 10, the minimum distance among the distances between the outer lines of the implant to be placed and the adjacent objects of the implant to be placed may not be the uppermost distance. If the minimum distance between the outer lines of the implant to be placed and the adjacent object of the implant to be placed is not the uppermost distance, an adjustable region 480 may be a region spaced apart from the outer contour line of the adjacent object of the implant to be placed by a clearance distance, which is a predefined reference value.

When the minimum distance between the outer lines of the implant to be placed and the adjacent object of the implant to be placed is determined to be other than the uppermost distance, it may be considered that a degree of freedom of implant adjustment is high in performing implant placement. Therefore, the implant placement operator may be provided with a maximum width of the adjustable region so as to be ensured of a degree of adjustment.

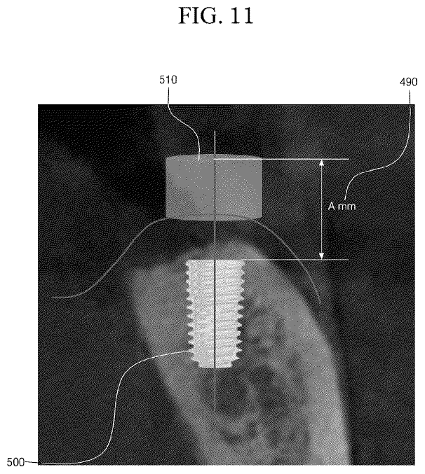

Next, an operation of determining an adjustable region by using a crown region of an adjacent tooth of an implant to be placed will be described with reference to FIGS. 11 to 13.

FIG. 11 illustrates an example of a required guide drilling hole region 510 according to a minimum hole height 490 necessary for placement of an implant 500. A height of the required guide drilling hole region 510 may be determined by reflecting the minimum hole height 490 of A mm required for placement of the implant 500. In one embodiment, the height of the required guide drilling hole region 510 is a height determined according to a specification of a drill product for placing the implant 500.

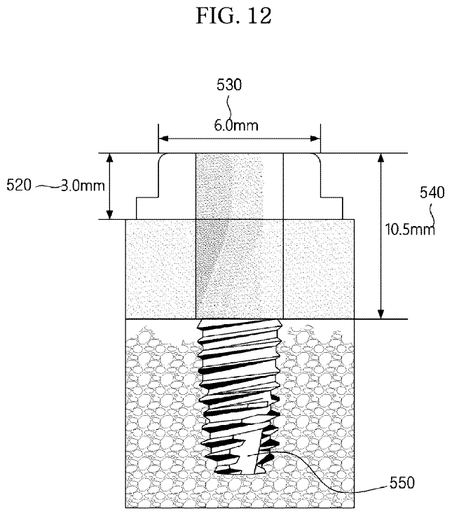

FIG. 12 illustrates an example of a required guide drilling hole region consisting of a height 520 of the required guide drilling hole region and a diameter 530 of the required guide drilling hole region for placement of an implant 550. The height 520 of the required guide drilling hole region may be determined according to a specification of a drill product for placing the implant 550.

As illustrated, for example, when the height 520 of the required guide drilling hole region is 3.0 mm and the diameter 520 of the required guide drilling hole region is 6.0 mm, a minimum hole height 540 required for placing the implant 550 may be set to 10.5 mm.

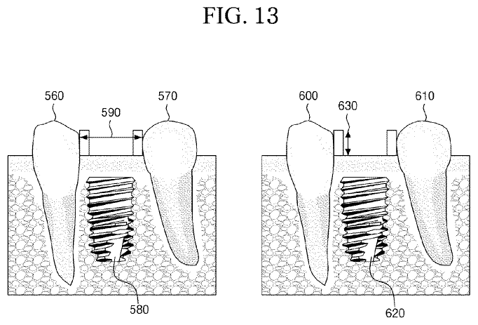

Next, with reference to FIG. 13, an operation of determining whether a space provided by adjacent teeth of implants 580 and 620 to be placed conforms to a required guide drilling hole region will be described.

FIG. 13 illustrates a minimum distance 590 between adjacent teeth of a guide drilling hole and a minimum height 630 of a guide drilling hole for creation of the guide drilling hole.

When a crown-provided diameter, which is a distance between a left-tooth crown 560 and a right-tooth crown 570 adjacent to the implant 580 to be placed, has a value greater than or equal to the minimum distance 590 between adjacent teeth of the guide drilling hole, it may be determined that the space conforms to the required guide drilling hole region.

In addition, when a crown-provided height set based on a left-tooth crown 600 and a right-tooth crown 610 adjacent to the implant 620 to be placed has a value greater than or equal to the minimum height 630 of the guide drilling hole, it may be determined that the space conforms to the required guide drilling hole region.

The minimum distance 590 between adjacent teeth and the minimum height 630 are minimum criteria for normal creation of the guide drilling hole. If the corresponding diameter and height fail to include the guide drilling hole region and thus creation of the guide drilling hole is not ensured, a drilling hole may not be created, or an error may occur during implant placement.

Therefore, when the crown-provided diameter and the crown-provided height do not include the minimum distance 590 between adjacent teeth of the guide drilling hole and the minimum height 630 of the guide drilling hole, respectively, the position of the graphic object of the implant including the crown-provided region that includes the guide drilling hole must be re-determined through implant fine adjustment.

Accordingly, when the crown-provided diameter and the crown-provided height fail to include the minimum distance 590 between adjacent teeth of the guide drilling hole and the minimum height 630 of the guide drilling hole and thus creation of the guide drilling hole is not ensured, the implant fine-adjustment screen may be updated to an implant fine-adjustment screen in which the activation state of the manipulator included in the implant fine-adjustment screen is maintained.

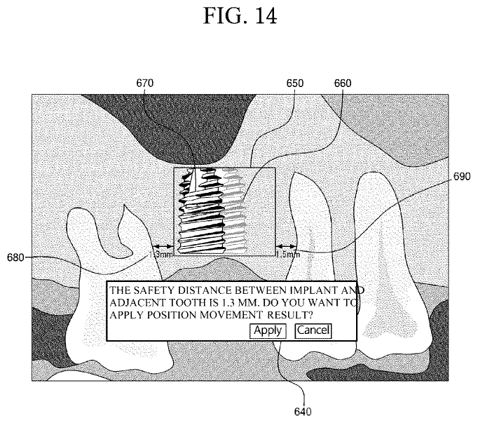

Next, with reference to FIG. 14, an implant fine-adjustment screen in which information indicating that a graphic object of the implant deviates from an adjustable region graphic object upon reflection of an adjustment signal is visualized will be described. When the graphic object of the implant deviates from the adjustable region graphic object upon reflection of the adjustment signal, the implant fine-adjustment screen may be updated to include a deviation notification message 640. In addition, the implant fine-adjustment screen may be updated to include a color or shading of an adjustable region 650 that is expressed differently from other regions.

For example, among a spacing distance from a left tooth 680 and a spacing distance from a right tooth 690, a color or shading of an object indicating the spacing distance 680 from the left tooth 680 where the collision has occurred may be displayed differently from an object indicating the spacing distance from the right tooth, thereby emphasizing that the deviation has occurred on the left side.

When a graphic object of the implant deviates from the adjustable region graphic object upon reflection of the adjustment signal, an implant fine-adjustment screen including both an implant graphic object 660 reflecting previous adjustment conditions and an implant graphic object 670 reflecting the latest input adjustment conditions may be generated simultaneously.

On the implant fine-adjustment screen including both the implant graphic object 660 reflecting previous adjustment conditions and the implant graphic object 670 reflecting the latest input adjustment conditions, a selection to change the currently active implant graphic object to the implant graphic object 660 reflecting the previous adjustment conditions may be input.

So far, a method for providing an implant adjustment guide according to several embodiments of the present disclosure has been described in detail. It should be understood that the embodiments described above are illustrative in all aspects and are not limiting.

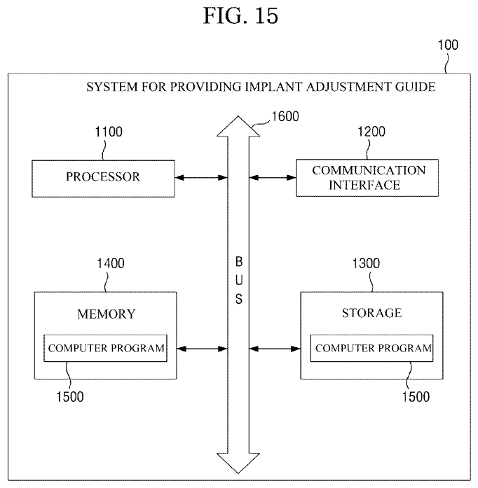

FIG. 15 is a hardware configuration diagram of an implant adjustment guide providing system according to some embodiments of the present disclosure. The implant adjustment guide providing system 100 of FIG. 15 may include at least one processor 1100, a system bus 1600, a communication interface 1200, a memory 1400 configured to load a computer program 1500 executed by the processor 1100, and a storage 1300 configured to store the computer program 1500.

The processor 1100 controls the overall operation of each configuration of the implant adjustment guide providing system 100. The processor 1100 may perform computations for at least one application or program for executing methods and/or operations according to various embodiments of the present disclosure. The memory 1400 stores various types of data, commands, and/or information. The memory 1400 may load one or more computer programs 1500 from the storage 1300 in order to execute the methods and/or operations according to various embodiments of the present disclosure. The bus 1600 provides communication functionality between the components of the implant adjustment guide providing system 100. The communication interface 1200 supports Internet communication of the implant adjustment guide providing system 100. The storage 1300 may non-temporarily store one or more computer programs 1500. The computer programs 1500 may include one or more instructions implementing the methods and/or operations according to various embodiments of the present disclosure. When the computer program 1500 is loaded on the memory 1400, the processor 1100 may execute the one or more instructions to perform the methods and/or operations according to various embodiments of the present disclosure.

In some embodiments, the implant adjustment guide providing system 100 described with reference to FIG. 15 may be configured using one or more physical servers included in a server farm, based on cloud technologies, such as virtual machines. In this case, some of the components depicted in FIG. 15, such as the processor 1100, the memory 1400, and the storage 1300, may be implemented as virtual hardware. Similarly, the communication interface 1200 may be composed of virtualized networking elements, such as a virtual switch.

The computer program 1500 may include an instruction for obtaining an oral cavity shape of an implant subject undergoing implant placement, an instruction for determining an initial position of a graphic object of an implant, an instruction of determining an adjustable region for the graphic object of the implant by using the oral cavity shape, wherein the adjustable region, which is a region adjacent to the initial position of the implant, is a maximum region where, according to the oral cavity shape, no objects are present in an oral cavity and is displayed at a fixed position regardless of positional movement of the graphic object of the implant, an instruction for providing an implant fine-adjustment screen including the graphic object of the implant and an adjustable region graphic object in which the determined adjustable region is visualized, an instruction for receiving input of an implant adjustment signal generated by a user input to a user interface included in the implant fine-adjustment screen, an instruction for moving a graphic object position of the implant in response to the implant adjustment signal, and an instruction for outputting a predefined user confirmation message when the moved graphic object position of the implant has deviated from the adjustable region.

Various embodiments of the present disclosure and effects thereof have been described with reference to FIGS. 1 to 15 so far. Effects according to the technical features of the present disclosure are not limited to the above-described effects, and other effects not described will be clearly understood by those skilled in the art from the following description.

The technical features of the present disclosure described above may be implemented as computer readable codes on a computer-readable medium. The computer program recorded on the computer-readable recording medium may be transmitted to another computing device through a network such as the Internet and installed in the other computing device, thereby being used in the other computing device.

Although the operations are shown in a specific order in the drawings, it should not be understood that the operations must be performed in the specific order or sequential order shown, or that all the illustrated operations must be executed to achieve the desired results. In certain circumstances, multitasking and parallel processing may be advantageous. Although the embodiments of the present disclosure have been described with reference to the attached drawings, those skilled in the art will appreciate that many variations and modifications can be made to the exemplary embodiments without substantially departing from the principles of the present disclosure. Therefore, the disclosed exemplary embodiments of the invention are used in a generic and descriptive sense only and not for purposes of limitation. The scope of the present disclosure should be interpreted by the claims below, and any technical features within the scope equivalent to the claims should be interpreted as falling within the scope of the technical features defined by the present disclosure.

1. A method for providing an implant adjustment guide, the method being performed by a computing device, the method comprising the steps of:
obtaining an oral cavity shape of a subject undergoing implant placement;
determining, by using the oral cavity shape, an initial position of a graphic object of an implant;
determining, by using the oral cavity shape, an adjustable region for the graphic object of the implant,
wherein the adjustable region, which is a region adjacent to the initial position of the implant, is a maximum region where, according to the oral cavity shape, no object is present inside the oral cavity, and is a region displayed at a fixed position regardless of positional movement of the graphic object of the implant; and
providing an implant fine-adjustment screen including the graphic object of the implant and an adjustable region graphic object in which the determined adjustable region is visualized.

2. The method of claim 1, wherein the step of determining, by using the oral cavity shape, an adjustable region for the graphic object of the implant comprises the step of obtaining a region spaced apart from an intraoral object by a reference value in the oral cavity shape.

3. The method of claim 2, wherein the step of obtaining a region spaced apart from an intraoral object by a reference value in the oral cavity shape comprises the steps of:
obtaining a shape of the intraoral object; and
obtaining a region spaced apart from an outer contour line of the intraoral object by a predefined reference value.

4. The method of claim 3, wherein the step of obtaining a region spaced apart from an outer contour line of the intraoral object by a predefined reference value comprises the step of, when an uppermost distance between the outer contour line of the intraoral object and the implant to be placed is minimum, determining, as a maximum width of the adjustable region, a distance obtained by excluding a distance that must not be invaded by the implant to be placed from the uppermost distance.

5. The method of claim 1, wherein the step of providing an implant fine-adjustment screen including the graphic object of the implant and an adjustable region graphic object in which the determined adjustable region is visualized comprises the steps of:
receiving an implant adjustment signal generated by a user input to a user interface included in the implant fine-adjustment screen;
moving a graphic object position of the implant in response to the implant adjustment signal; and
outputting a predefined user confirmation message when the moved graphic object position of the implant has deviated from the adjustable region.

6. The method of claim 5, wherein the step of moving a graphic object position of the implant in response to the implant adjustment signal comprises the step of, when the moved graphic object position of the implant has deviated from the adjustable region, providing an implant fine-adjustment screen in which the graphic objects of the implant are respectively arranged at a position before movement and a position after movement according to the implant adjustment signal.

7. The method of claim 6, wherein the step of providing an implant fine-adjustment screen in which the graphic objects of the implant are respectively arranged at a position before movement and a position after movement according to the implant adjustment signal comprises the step of providing the implant fine-adjustment screen such that the graphic object of the implant at the position after movement according to the implant adjustment signal is overlaid on the graphic object of the implant at the position before movement.

8. The method of claim 6, wherein the step of, when the moved graphic object position of the implant has deviated from the adjustable region, providing an implant fine-adjustment screen in which the graphic objects of the implant are respectively arranged at a position before movement and a position after movement according to the implant adjustment signal comprises the step of generating a selection message regarding whether to apply the implant adjustment signal.

9. The method of claim 6, wherein the step of, when the moved graphic object position of the implant has deviated from the adjustable region, providing an implant fine-adjustment screen in which the graphic objects of the implant are respectively arranged at a position before movement and a position after movement according to the implant adjustment signal comprises the step of changing a region expression element of the graphic object of the implant.

10. The method of claim 5, wherein the step of moving the graphic object position of the implant in response to the implant adjustment signal comprises the steps of:
obtaining a required guide drilling hole region;
obtaining a crown region of an adjacent tooth of the implant in the oral cavity shape;
obtaining a region in the oral cavity shape which is spaced apart from the crown region by a reference value and in which the required guide drilling hole region is entirely included; and
selecting, as an implant adjustable region, the region in the oral cavity shape which is spaced apart from the crown region by the reference value and in which the required guide drilling hole region is entirely included.

11. A system for providing an implant adjustment guide, comprising:
a memory into which an implant adjustment guide providing program is loaded; and
a processor configured to execute the implant adjustment guide providing program,
wherein the implant adjustment guide providing program comprises:
an instruction for obtaining an oral cavity shape of a subject undergoing implant placement;
an instruction for determining an initial position of a graphic object of the implant using the oral cavity shape;
an instruction for determining an adjustable region for the graphic object of the implant using the oral cavity shape, wherein the adjustable region, which is a region adjacent to the initial position of the implant, is a maximum region where, according to the oral cavity shape, no object is present inside the oral cavity, and is a region displayed at a fixed position regardless of positional movement of the graphic object of the implant;
an instruction for providing an implant fine-adjustment screen including the graphic object of the implant and an adjustable region graphic object in which the determined adjustable region is visualized;
an instruction for receiving an implant adjustment signal generated by a user input to a user interface included in the implant fine-adjustment screen;
an instruction for moving a graphic object position of the implant in response to the implant adjustment signal; and
an instruction for outputting a predefined user confirmation message when the moved graphic object position of the implant has deviated from the adjustable region.
